(19) United States
(12) Patent Application Publication
(10) Pub. No.: US 2012/0148643 A1
(43) Pub. Date: Jun. 14, 2012

(54) TOOTH BLEACHING CATALYTIC AND APPLICATION THEREOF

(75) Inventors: Chia-Wen WU, Taipei (TW); Bor-Shiunn Lee, Taipei (TW); Li-Chun Huang, Taipei (TW); Chun-Pin Lin, Taipei (TW)

(73) Assignee: National Taiwan University, Taipei (TW)

(21) Appl. No.: 13/024,667

(22) Filed: Feb. 10, 2011

(30) Foreign Application Priority Data
Dec. 9, 2010 (TW) 099143041

(51) Int. Cl. A61K 8/02; A61K 8/44; A61K 8/22; A61Q 11/00; B82Y 30/00

(52) U.S. Cl. 424/401; 424/53; 424/54; 977/919

(57) ABSTRACT

A tooth bleaching catalytic, the manufacturing method and the applications thereof are provided, wherein the tooth bleaching catalytic comprises a plurality of mesoporous silica nano-particles (MSNs), and the MSNs at least comprise a condensate having histidine, silane and a plurality of metal ions.

### BACKGROUND

1. Technical Field

Present invention is related to a tooth bleaching catalytic and the applications thereof, more particularly related to a light-free tooth bleaching catalytic and the applications thereof.

2. Description of the Related Art

Tooth discoloration can negatively impact self-image and self-confidence. Generally, extrinsic stains (tooth discoloration) can easily be corrected through routine prophylactic procedures, micro-abrasion or macro-abrasion Improvement of intrinsic discoloration, however, requires tooth bleaching.

Materials currently used for tooth bleaching, such as sodium perborate or carbamide peroxide, are based on released hydrogen peroxide (H₂O₂) there from serves as active agent to bleach discolored teeth. Nevertheless, the H₂O₂-based tooth bleaching agents have limits on bleaching severely discolored teeth.

A thermo-catalytic technique, which consists of applying heat or light (LED or laser) to activate the bleaching agent, is frequently used to enhance the bleaching efficiency of these materials. However, applying heat or light may cause problems of cervical root resorption. Since cervical root resorption is usually asymptomatic and is only detected by sporadic radiographic examination, thus it is hard to get an early diagnosis and treatment, and a tooth extraction may be required in the case of the formation of large lesions.

Therefore, it is necessary to provide an improved tooth bleaching catalytic and the applications thereof to avoid this possible adverse effect of the traditional bleaching.

### BRIEF SUMMARY

One aspect of the present invention is to provide a tooth bleaching catalytic comprising a plurality of mesoporous silica nano-particles (MSNs), wherein the MSNs at least comprise a condensate having histidine, silane and a plurality of metal ions.

In some embodiments of the present invention, the plurality of ions are selected from the group consisting of cupric ions (Cu(II)), ferrous ions(Fe(II)), manganese ion (Mn(II)) and the arbitrary combination thereof.

In some embodiments of the present invention, the MSNs comprise substantially 1% of the plurality of ions by weight.

In some embodiments of the present invention, the MSNs have an average diameter substantially ranges from 50 nm to 100 nm.

In some embodiments of the present invention, the MSNs have a wormlike shape.

In some embodiments of the present invention, the tooth bleaching agent activated by the tooth bleaching catalytic is H₂O₂.

Another aspect of the present invention is to provide a tooth bleaching method comprising steps as follows: First, a tooth bleaching agent and a tooth bleaching catalytic having a plurality of MSNs are provided, wherein the MSNs at least comprise a condensate having histidine, silane and a plurality of metal ions. Subsequently, the tooth bleaching catalytic is mixed with the tooth bleaching agent, and the tooth bleaching agent mixed with the tooth bleaching catalytic is used in contact with at least one discolored tooth.

In some embodiments of the present invention, the tooth bleaching agent is H₂O₂.

In some embodiments of the present invention, the preparation of the tooth bleaching catalytic further comprises preparing at least one histidine-mesoporous silica nano-particle (histidine-MSN) and a plurality of metal ions, and comdensating the plurality of metal ions with the histidine-MSN.

In some embodiments of the present invention, the plurality of ions are selected from the group consisting of Fe(II), Mn(II), and Cu(II) and the arbitrary combination thereof.

In accordance with the embodiments of the present invention, a tooth bleaching catalytic comprising a plurality of MSNs having histidine, silane and a plurality of metal ions is provided to activate the tooth bleaching agent (such as H₂O₂) to bleach discolor tooth without applying heat or light, whereby the adverse effect of bleaching due to the application of heat and light can be avoided.

In order to make the aforementioned and other objects, features and advantages of the present invention comprehensible, preferred embodiments accompanied with figures are described in detail below. Other objectives, features and advantages of the present invention will be further understood from the further technological features disclosed by the embodiments of the present invention wherein there are shown and described preferred embodiments of this invention, simply by way of illustration of modes best suited to carry out the invention.

### BRIEF DESCRIPTION OF THE DRAWINGS

These and other features and advantages of the various embodiments disclosed herein will be better understood with respect to the following description and drawings, in which like numbers refer to like parts throughout, and in which:

FIGS. 1A to 1C are photographs of the present tooth bleaching catalytic and other comparison specimens taken by a scanning electron microscopy (SEM), in accordance with some preferred embodiments of present invention.

FIG. 2 illustrates the diffraction patterns of the present tooth bleaching catalytic and other comparison specimens by utilizing an X-ray diffraction (XRD), in accordance with some preferred embodiments of present invention.

FIGS. 3A to 3D are images of the present tooth bleaching catalytic and other comparison specimens taken by a transmission electron microscopy (TEM), in accordance with some preferred embodiments of present invention.

FIGS. 4A to 4C illustrate color difference histograms of the color difference examination respectively conducted on the enamel, the outer dentin and the inner dentin in accordance with the results of the bleaching test of stained tooth.

FIG. 5 illustrates photographs of the tooth specimens observed during the bleaching test of stained tooth taken by a stereoscopic microscope.

### DETAILED DESCRIPTION

The object of the present invention is to provide a tooth bleaching catalytic, the manufacturing method and the applications thereof, by which the problems of cervical root resorption due to the adverse effect of the heat and light that are applied to activate the traditional bleaching agent can be avoided.

In some embodiments of the present invention, the tooth bleaching catalytic comprises a plurality of MSNs, wherein the MSNs at least comprise a condensate having histidine, silane and a plurality of metal ions.

To describe the make, use and applications of the present tooth bleaching catalytic, several preferred embodiments of syntheses of the tooth bleaching catalytic are described in detail below. Subsequently, an X-ray diffraction (XRD) and a scanning electron microscopy (SEM) are utilized to investigate and characterize the morphology of the present tooth bleaching catalytic, and a tooth bleaching test of stained tooth is conducted to evaluate the catalytic ability of the present tooth bleaching catalytic.

□. Synthesis of the Tooth Bleaching Catalytic

The method for manufacturing the tooth bleaching catalytic comprises steps as follows: First, at least one histidine-MSN is prepared by following procedures. In the present embodiment, triethylamine (0.966 mL, 9.5 mmol) and ethyl chloroformate (0.55 mL, 50 mmol) are added into a cooled (0° C.) solution of Di-boc-histidine (1 g, 2.7 mmol) in chloroform (15 mL) and stirred for 15 minutes. Subsequently (3-aminopropyl) triethoxysilane (APTES) (1 mL, 4.5 mmol) and triethylamine (0.966 mL, 9.5 mmol) are added, and the solution is stirred at 0° C. for 90 minutes. The cold mixture is then added to dichloromethane, and the solvent is removed using a rotatory evaporator to obtain a histidine-containing silane.

A surfactant cetyltrimethylammonium bromide (CTAB, 1.0 g) and NaOH (2N, 3.5 mL) are added into distilled water (480 mL), and the mixture is heated to 80° C. To this solution, the histidine-containing silane (4.2 mmole in 0.5 mL of chloroform) is added before the addition of tetraethoxysilane (TEOS, 5 mL). Both histidine-silane and TEOS are added drop by drop at a rate of 0.5 mL/min After undergoing reaction at 80° C. for 2 hours, the white precipitate is collected and dried in a vacuum, whereby a histidine-functionalized MSN is obtained.

After removing the surfactant, the histidine-functionalized MSN (1.0 g) is added to a solution containing 1.0 mL concentrated HCI and 150 mL methanol, followed by refluxing at 60° C. for 6 hours. The surfactant-free material, named as His-MSNs, is washed with methanol and distilled water, and then dried in a vacuum.

Subsequently, a condensation is conducted by which metal ions including Fe(II), Mn(II), and Cu(II) are incorporated into the His-MSNs by immersing 0.5 g of His-MSNs into 150 mL of various aqueous solutions containing different metal ions under stir at room temperature for 24 hours. In the some embodiments of the present invention, the aqueous solutions can be metal chlorides, such as FeCl₂.4H₂O, Mn(NO₃)₂.xH₂O, CuC1₂.2H₂O or the arbitrary combinations thereof (wherein the concentrations of metal chlorides are all kept at 19.2 mmol). The samples are washed with distilled water and dried in a vacuum, such that the present tooth bleaching catalytic named as M-his-MSN (wherein M is referred as Fe(II), Mn(II), and Cu(II)) are obtained.

□. Morphology Analysis of the Tooth Bleaching Catalytic

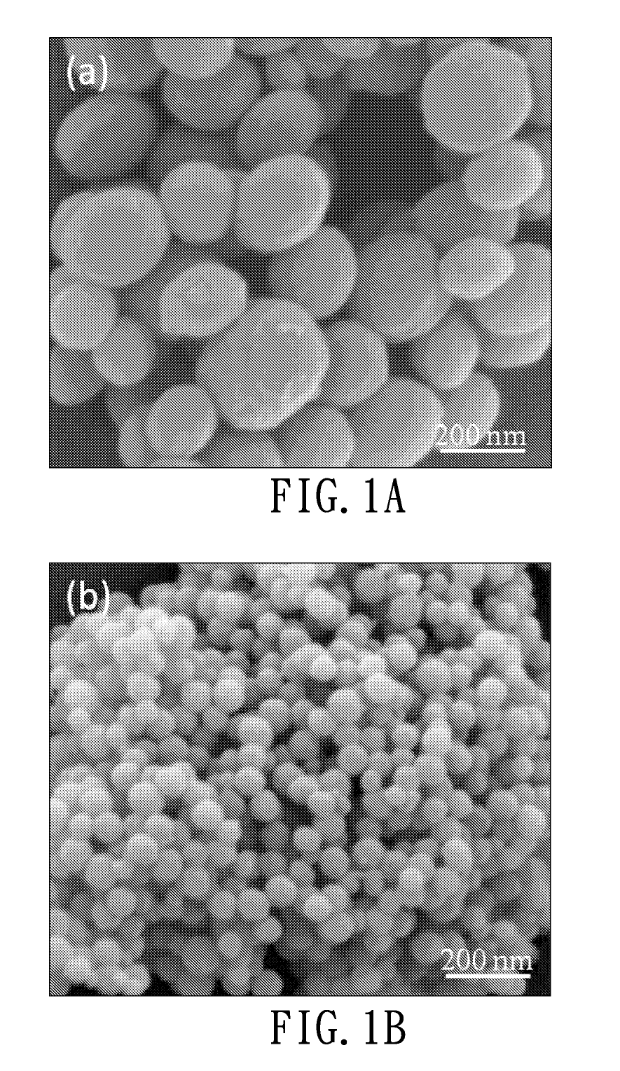

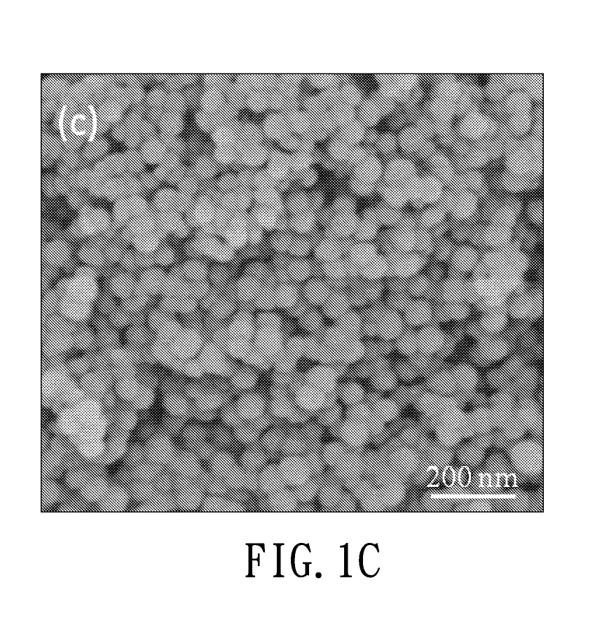

An XRD and a SEM are then utilized to investigate and characterize the morphology of the tooth bleaching catalytic. FIGS. 1A to 1C are photographs of the present tooth bleaching catalytic and other comparison specimens taken by a SEM, in accordance with some preferred embodiments of present invention. FIG. 1A is a SEM photograph of histidine and metal ion free MSN (serves as a control); FIG. 1B is a SEM photograph of His-MSNs; and FIG. 1C is a SEM photograph of Fe(II)-his-MSNs, wherein the scale bars of the FIGS. 1A, 1B and 1C indicate 200 nm. The average diameter of the specimens can be estimated in accordance with the scale bar of the SEM photographs. In the present embodiment, Fe(II)-his-MSNs has an average diameter substantially ranges from 50 nm to 100 nm. In comparison with the MSNs and the His-MSNs (free from comdensating with metal ion), the average diameter of the Fe(II)-his-MSNs is smaller than that of the MSN and the His-MSN. However, it should be appreciated that the average diameter of the Fe(II)-his-MSNs is just an example, the average diameter of the M-his-MSN may vary depends upon what kind of metal ions, such as Fe(II), Mn(II), Cu(II) or the arbitrary combinations thereof, condensate with the His-MSNs. In sum, the average diameter of the M-his-MSNs substantially ranges from 50 nm to 100 nm.

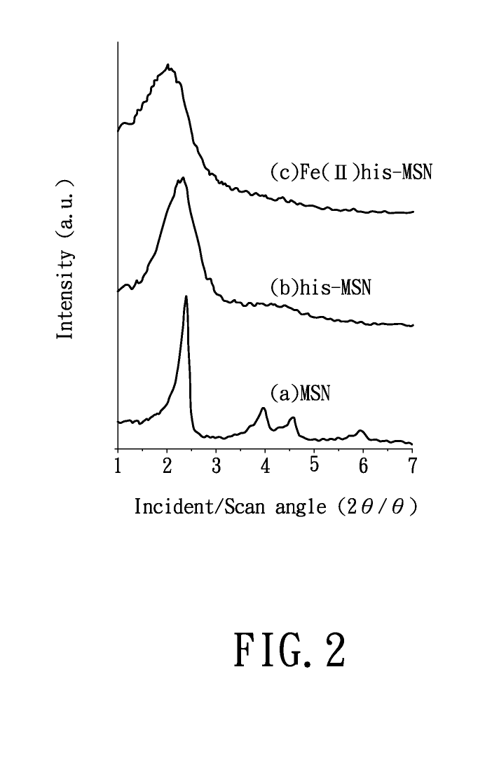

FIG. 2 illustrates the diffraction patterns of the tooth bleaching catalytic and other comparison specimens by utilizing an XRD, in accordance with some preferred embodiments of present invention. Wherein the curve (a) represents the diffraction pattern of the MSNs; the curve (b) represents the diffraction pattern of the His-MSNs; and the curve (c) represents the diffraction pattern of the Fe(II)-his-MSNs. The X-axis of the diffraction pattern indicates the incident/scan angle (2θ/74 ) of the X-ray, and the Y-axis indicates the detecting intensity (a.u.) of the incident X-ray beam. In comparison these three diffraction patterns, only a single broad peak is observed in the curve (c), from which it is concluded that the Fe(II)-his-MSN exhibits a worm-like mesostructure, uniform particle morphology, and large surface area. This conclusion can be verified by a transmission electron microscopy (TEM) investigation.

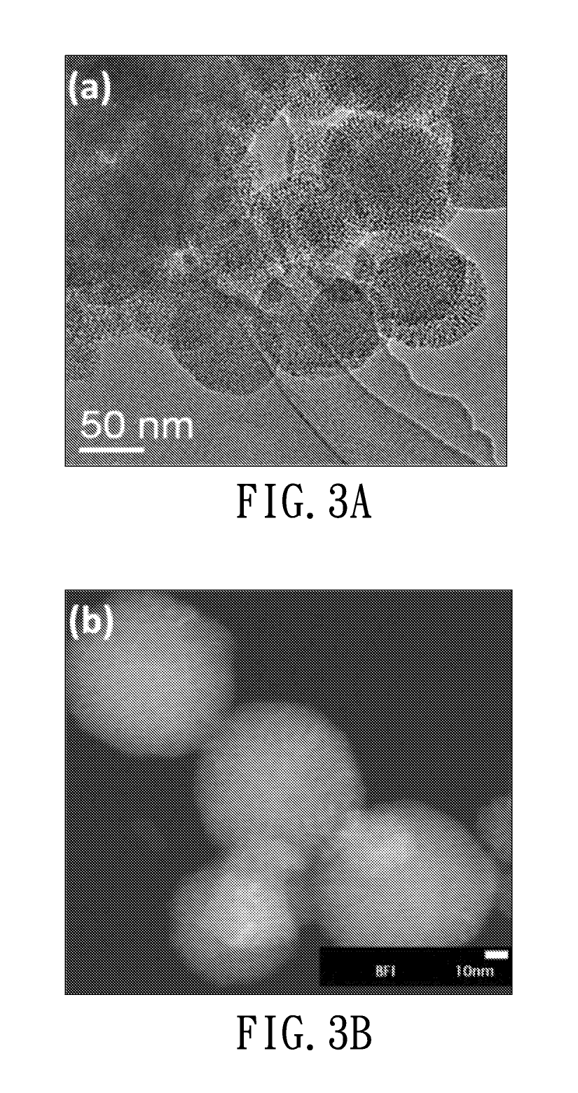

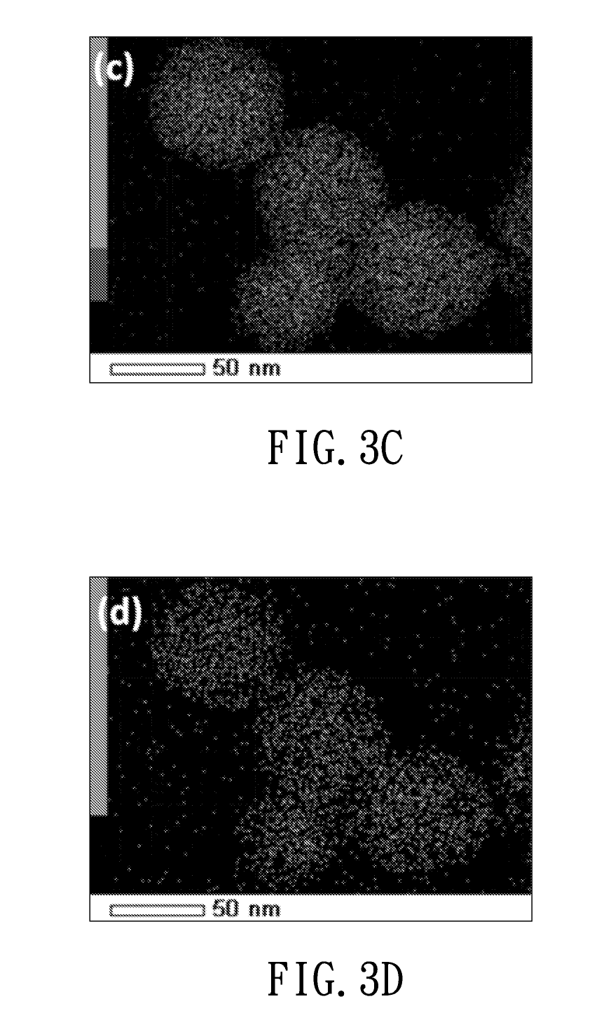

FIGS. 3A to 3D are images of the present tooth bleaching catalytic and other comparison specimens taken by a TEM, in accordance with some preferred embodiments of present invention. FIG. 3A is a bright field TEM image of the Fe(II)-his-MSNs; FIG. 3B is a dark field TEM image of the Fe(II)-his-MSNs; FIG. 3C is a high resolution elemental mapping of Si element in the Fe(II)-his-MSNs; and FIG. 3D is a high resolution elemental mapping of Fe element in the Fe(II)-his-MSNs, wherein the scale bars of the FIGS. 3A, 3B, 3C and 3D indicate 50 nm. Through a direct investigation of FIGS. 3A to 3D, it can be approved that the Fe(II)-his-MSNs content metal ions Fe(II) uniformly distributed inside the Fe(II)-his-MSN, and Fe(II)-his-MSN exhibits a worm-like mesostructure. In the present embodiment, the Fe(II)-his-MSNs have a surface area substantially about 1145 m²/g.

□. Bleaching Test of Stained Tooth-Extracted Tooth Model

(1) Testing Materials

A. Sample preparing:

A total of 15 extracted permanent molars are provided. Each molar is evenly sectioned into three pieces. These three pieces were then randomly assigned to one of three groups, such that each group eventually has 15 pieces.

B. Staining agent preparing: Orange II is diluted with distilled water to a concentration of 0.15 mM solution.

C. Tooth bleaching agent preparing: three tooth bleaching agents, denominated as Test 1, Test 2 and Control, are prepared, and the contents of the three tooth bleaching agents are set forth as follows:

Test 1: solution with 30% H₂O₂ by weight and containing the Fe(II)-his-MSNs serve as catalytic.

Test 2: solution with 30% H₂O₂ by weight and containing the Mn(II)-his-MSNs serve as catalytic.

Control: solution with 30% H₂O₂ by weight.

(2) Testing Procedures

A tooth staining process is firstly conducted; each of the tooth specimens is immersed in 10 ml of the staining agent (the Orange II solution) for 48 hours.

These three groups of the stained tooth specimens are then respectively immersed in 10 ml of the three different tooth bleaching agent. After each immersion period for 1, 3, 6, and 12 hours, the tooth specimens are removed and examined for color difference (ΔE*), meanwhile the bleaching agents are refreshed and then the tooth specimens are replaced in the refreshed bleaching agents. Wherein the color difference examinations are conducted on the enamel, the outer dentin and the inner dentin of the each examined tooth specimens.

The overall color difference (ΔE*) of the specimens was calculated based on International Commission on Illumination (CIE) Lab system using the following formula:

ΔE*=\sqrt{(ΔL*)²+(Δa*)²+(Δb*)²}

Wherein the L* value represents the degree of lightness within a sample and ranges from 0 (black) to 100 (white), the a* value detects the degree of greenness (negative a*) or redness (positive a*), and the b* value measures the degree of blueness (negative b*) or yellowness (positive b*) of the sample. Since the color difference evaluation is based on International Commission on Illumination (CIE) Lab system, and the procedure of which has been well known by those skilled in the art, thus the specification hereinafter will not describe the operating steps thereof in detail.

Significant differences in color difference (ΔE*) within various conditions were analyzed using a one-way analysis of variance (one-way ANOVA) followed by least squares means test. A value of p<0.05 was considered to represent statistically significant difference between tested data sets.

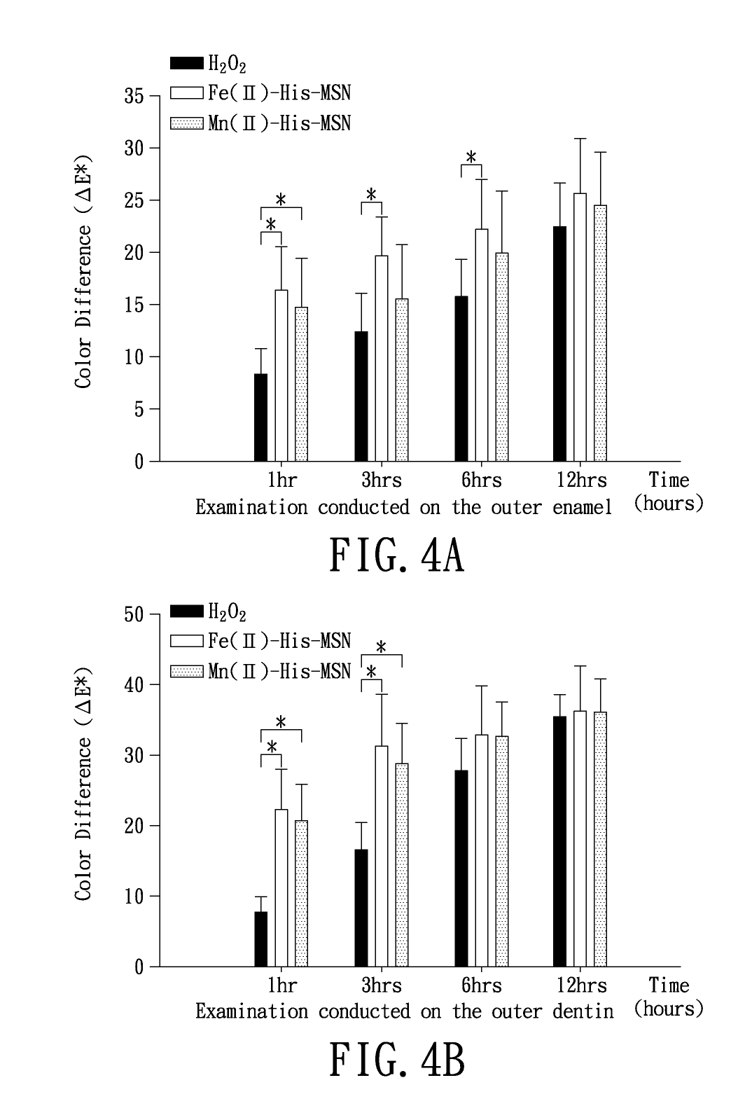

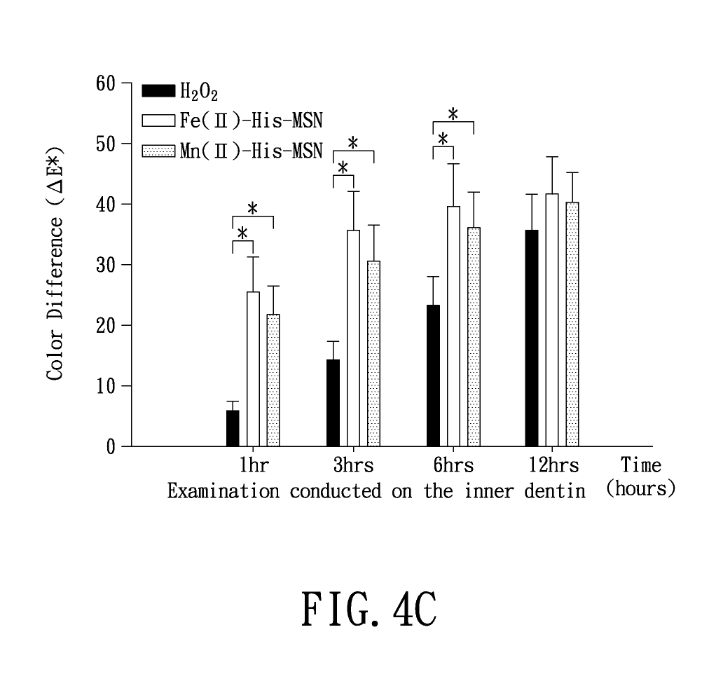

The results of the bleaching test are illustrated in FIGS. 4A to 4C. Wherein FIG. 4A illustrates a color difference histogram of the color difference examination conducted on the enamel; FIG. 4B illustrates a color difference histogram of the color difference examination conducted on the outer dentin and FIG. 4C illustrates a color difference histogram of the color difference examination conducted on the inner dentin. The X-axis of the histograms indicate the immersion period, and the Y-axis indicate the color difference (ΔE*) of the specimens. FIGS. 4A to 4C reveal that there are significant difference in bleaching ability between the Control bleaching agent and the two tooth bleaching agents. The bleaching agent Test 1 containing the Fe(II)-his-MSNs has the best bleaching ability, the bleaching agent Test 2 containing the Mn(II)-his-MSNs has an inferior bleaching ability inferior to that of the bleaching agent Test 1, and both of them have better bleaching ability than the Control bleaching agent.

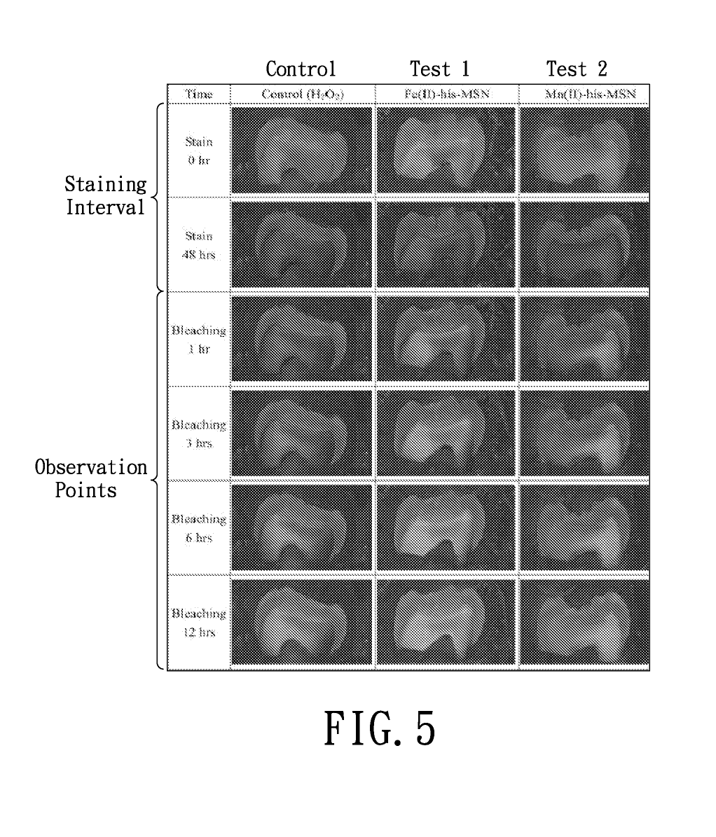

FIG. 5 illustrates photographs of the tooth specimens observed during the bleaching test of stained tooth taken by a stereoscopic microscope. FIG. 5 is divided in 3 columns and each column comprises 6 photographs of the tooth specimens. The tooth specimens of the three columns are respectively bleached by the three different bleaching agents, Test 1, Test 2 and Control, and the 6 photographs of each column are the images taken at various observation points of 1st, 3rd, 6th, and 12th hours. In comparison to the images of FIG. 5, it reveals that the bleaching agent Test 1 containing the Fe(II)-his-MSNs has the best bleaching ability, the bleaching ability of the bleaching agent Test 2 containing the Mn(II)-his-MSNs is inferior to that of the bleaching agent Test 1, and both of them have better bleaching ability than the Control bleaching agent free from applying any catalytic. It can be concluded that present M-his-MSNs can be used as tooth bleaching catalytic to activate the tooth bleaching agent rather than applying heat or light.

In accordance with the embodiments of the present invention, a tooth bleaching catalytic comprising a plurality of MSNs having histidine, silane and a plurality of metal ions is provided to activate the tooth bleaching agent (such as H₂O₂) free from applying heat or light to bleach discolor tooth, whereby the adverse effect of bleaching due to the application of heat and light can be avoided.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A tooth bleaching catalytic, comprising a plurality of mesoporous silica nano-particles (MSNs), wherein the MSNs at least comprise a condensate having histidine, silane and a plurality of metal ions.

2. The tooth bleaching catalytic as claimed in claim 1, wherein the plurality of ions are selected from the group consisting of cupric ions (Cu(II)), ferrous ions(Fe(II)), manganese ion (Mn(II)) and the arbitrary combination thereof.

3. The tooth bleaching catalytic as claimed in claim 1, wherein the MSNs comprise substantially 1% of the plurality of ions by weight.

4. The tooth bleaching catalytic as claimed in claim 1, wherein the MSNs have an average diameter substantially ranges from 50 nm to 100 nm.

5. The tooth bleaching catalytic as claimed in claim 1, wherein the MSNs have a wormlike shape.

6. The tooth bleaching catalytic as claimed in claim 1, wherein the tooth bleaching catalytic is used to activate a tooth bleaching agent of H₂O₂.

7. A tooth bleaching method, comprising steps as follows:
providing a tooth bleaching agent and a tooth bleaching catalytic having a plurality of MSNs, wherein the MSNs at least comprise a condensate having histidine, silane and a plurality of metal ions;
mixing the tooth bleaching catalytic with the tooth bleaching agent; and
using the tooth bleaching agent mixed with the tooth bleaching catalytic in contact with at least one discolored tooth.

8. The tooth bleaching method as claimed in claim 7, wherein the tooth bleaching agent is H₂O₂.

9. The tooth bleaching method as claimed in claim 7, wherein the preparation of the tooth bleaching catalytic comprises steps as follows:
preparing at least one histidine-mesoporous silica nano-particle (histidine-MSN), and a plurality of metal ions; and
comdensating the plurality of metal ions with the histidine-MSN.

10. The tooth bleaching method as claimed in claim 9, wherein the plurality of ions are selected from the group consisting of Fe(II), Mn(II), and Cu(II) and the arbitrary combination thereof.

11. The tooth bleaching method as claimed in claim 10, wherein the condensation step comprises incorporating the metal ions into the His-MSN by immersing the His-MSN into an aqueous solution containing the metal ions under stir at room temperature for 24 hours.

12. The tooth bleaching method as claimed in claim 11, wherein the aqueous solution comprising a metal chloride.

13. The tooth bleaching method as claimed in claim 11, wherein the metal chloride is FeCl₂.4H₂O, Mn(N0₃)₂.xH₂O, CuCl₂.2H₂O or the arbitrary combinations thereof

14. The tooth bleaching method as claimed in claim 7, wherein the MSNs comprise substantially 1% of the plurality of ions by weight.

15. The tooth bleaching method as claimed in claim 7, wherein the MSNs have an average diameter substantially ranges from 50 nm to 100 nm.

16. The tooth bleaching method as claimed in claim 7, wherein the MSNs have a wormlike shape.
